(19) United States
(12) Patent Application Publication
(10) Pub. No.: US 2013/0060251 A1
(43) Pub. Date: Mar. 7, 2013

(54) PROXIMAL HUMERUS GREATER TUBEROSITY HOOK-ARM CLIP

(71) Applicant: Walter Andrew Eglseder, JR., Owings Mills, MD (US)

(72) Inventor: Walter Andrew Eglseder, JR., Owings Mills, MD (US)

(73) Assignee: University of Maryland, Baltimore, Baltimore, MD (US)

(21) Appl. No.: 13/601,688

(22) Filed: Aug. 31, 2012

Related U.S. Application Data
(60) Provisional application No. 61/529,631, filed on Aug. 31, 2011.

(51) Int. Cl. A61B 17/80; A61B 17/88

(52) U.S. Cl. 606/71; 606/289; 606/281

(57) ABSTRACT

The invention relates to a bone plate for stabilizing a fractured greater tuberosity, wherein the bone plate comprises one or more apertures for fixing the bone plate to bone; one or more hook-arm receivers; one or more attachable hook-arms comprising one or more prongs and a device for attachment to the bone plate. The invention also relates to methods for stabilizing a fractured greater tuberosity using the bone plates of the invention and kits comprising the same.

### CROSS REFERENCE TO RELATED APPLICATIONS

This application claims the benefit of U.S. Appl. No. 61/529,631, filed Aug. 31, 2011. The content of the aforesaid application is relied upon and incorporated by reference in its entirety.

### BACKGROUND OF THE INVENTION

1. Field of the Invention

The field of the invention relates generally to bone fracture fixation and, more particularly, to the fixation of bone fractures of the greater tuberosity.

2. Description of the Related Art

Fractures of the greater tuberosity present a unique problem because they often occur on the side of the humeral head making them difficult to fixate with currently available bone plate and suturing methods. In spite of the difficulty, it is very important to properly repair a greater tuberosity fracture. A superiorly displaced tuberosity can block abduction of the shoulder, whereas a posteriorly displaced tuberosity may impinge against the posterior glenoid and block external rotation. As such, failure to properly repair a greater tuberosity fracture can significantly impact a patient's quality of life.

Bone plates have been used to assist with bone fracture repair. They are produced in a variety of different designs and are commonly attached by screws and pins. Although internal fixation with bone plates and screws has been achieved, often times they cannot be used because the smaller fragments of the fracture site would not be able to withstand forces exerted by the screws. Bone plates have been developed with hooks at one end to fixate terminal bone fragments, such as a fragment of the lateral malleolus, medial malleolus, or proximal ulna as discussed in U.S. Patent Application Publication No. 2009/0275991. However, the hooks on these plates are rigidly attached to the end of an elongated body and therefore could not be used to fixate a fractured greater tuberosity.

To overcome the limitations of screws and pins, suturing methods have been developed to help reduce a fractured greater tuberosity. Sutures are commonly used in conjunction with a proximal humerus bone plate to help reduce greater tuberosity fragments. Bone plates for the proximal humerus typically have clips or suture holes around the edge of the bone plate head to aid in the suturing process. An alternative suturing approach is available which does not anchor to a bone plate. Here, the greater tuberosity fragment is reduced by suturing it to the supraspinatus and teres minor muscles. However, there are shortcomings with a suturing approach to reduce a fractured greater tuberosity because sutures may not provide adequate function for larger bone fragments.

The currently available devices and methods for securing a fractured greater tuberosity present undesirable limitations and there is a need for improved devices and methods to secure a fractured greater tuberosity.

This background information is provided for the purpose of making information believed by the applicant to be of possible relevance to the present invention. No admission is necessarily intended, nor should it be construed, that any of the preceding information constitutes prior art against the present invention.

### SUMMARY OF THE INVENTION

In one aspect, the invention provides a bone plate for stabilizing a fractured greater tuberosity of the proximal humerus, wherein the bone plate comprises one or more apertures for fixing the bone plate to the humerus bone; one or more hook-arm receivers in the bone plate; and one or more attachable hook-arms comprising one or more prongs and a device for attachment to the bone plate.

In one embodiment, the bone plate comprises a body portion at a first end attached to a head portion at a second end of the bone plate. In some embodiments, the receiver is on the head. In some embodiments, both the body and head portions comprise one or more apertures to fix the bone plate to the bone. In some embodiments, the apertures are configured to accommodate bone screws. In some embodiments, the body portion has an elongated shape, and has a width that is narrower than the head portion. In some embodiments, the head portion has a width that is enlarged relative to the body portion. In some embodiments, the hook-arm comprises a shaft having one or more prongs that project from the shaft located at the distal end of the shaft, and an attachment mechanism to the bone plate on the proximal end of the shaft. In some embodiments, the hook-arm shaft has a curvature along the central axis, j, as depicted in FIGS. 1 and 2, which can approximately match the curvature of the fracture site. In some embodiments, the one or more prongs is oriented at an angle of less than or equal to 90° to the distal end of the hook-arm shaft.

In some embodiments of the invention, the body portion of the bone plate comprises two or more bone screw apertures and the head portion has seven bone screw apertures. In some embodiments, one aperture is centrally located on the bone plate head portion while the remaining six apertures are evenly spaced around the outer edge of the head.

In some embodiments, the hook-arm has two prongs. In some embodiments, the hook-arm attachment mechanism is a ring at the proximal end of the hook-arm. In some embodiments, the ring can be inserted into a circular cavity located around one of the apertures of the head portion so that the hook-arm is flush with the rest of the bone plate. In some embodiments, the hook-arm is fixed in place with a bone screw that is inserted through the ring and screw aperture. In this embodiment, the hook-arm ring is like a washer between the bone screw head and the bone screw aperture.

In another aspect, the invention provides a method for stabilizing a fractured greater tuberosity in a subject comprising: i) attaching the bone plate of the invention to the humerus of a subject having a fractured greater tuberosity; ii) making one or more holes into the greater tuberosity; iii) securing the one or more prongs from the hook-arm into the holes of ii); iv) inserting the hook-arm attachment device into the hook-arm receiver; and v) securing the hook-arm to the bone plate, thereby stabilizing the fractured greater tuberosity.

In another aspect, the invention provides kits comprising one or more bone plates of the invention. In some embodiments, the kit comprises a plurality of hook-arms of different sizes. In some embodiments, the kit comprises a plurality of bone plates of different sizes and a plurality of hook arms of different sizes.

It is to be understood that both the foregoing general description of the invention and the following detailed description are exemplary, and thus do not restrict the scope of the invention.

### BRIEF DESCRIPTION OF THE DRAWINGS

The skilled artisan will understand that the drawings, described below, are for illustration purposes only. The drawings are not intended to limit the scope of the present teachings in any way.

FIG. 1 (A) is a front view of a single slot bone plate for a fractured greater tuberosity and (B) shows a hook-arm that can attach to the bone plate of (A).

FIG. 2 is a side view of a hook-arm for a fractured greater tuberosity.

FIG. 3 (A) shows a hook-arm receiver in a bone plate head portion with grooves to assist in the fixation of the hook-arm. (B) shows hook-arms in three different configurations with tongues or ridges that fit the grooves of the bone plate of (A).

FIG. 4 is a drawing of a bone plate with an extended hook-arm positioned on a model humerus showing its position on the greater tuberosity.

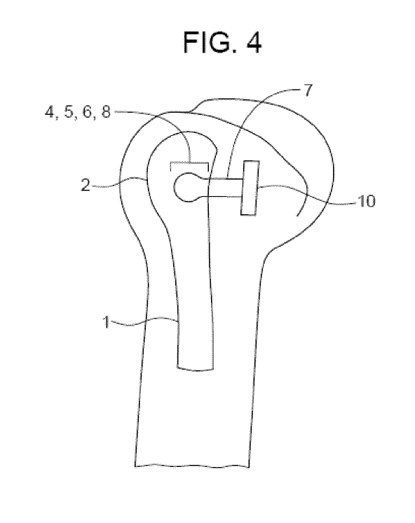

FIGS. 5A and B show a depiction of a bone plate with a hook-arm for securing a fracture of the greater tuberosity.

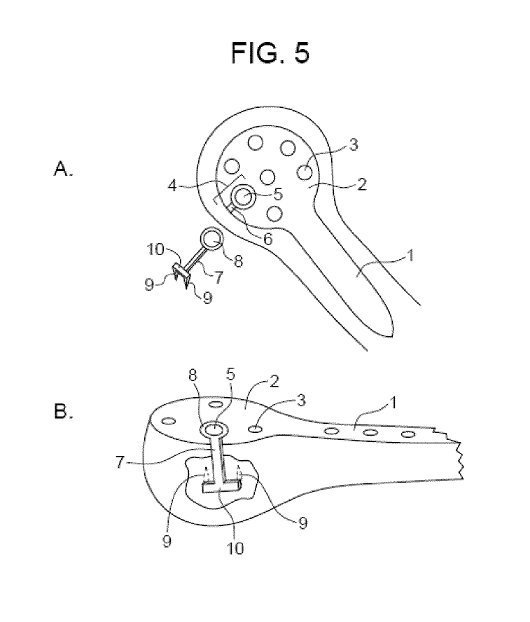

### DETAILED DESCRIPTION

The invention is based on the discovery that a fractured greater tuberosity of the proximal humerus can be secured with a bone plate with an attached hook-arm.

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of:” As used herein, “about” means±10% of the numerical value provided.

In one embodiment, the invention provides a bone plate comprising an upper surface and a bone contacting surface, capable of fixating a fractured greater tuberosity, wherein the bone plate comprises one or more apertures for fixing the bone plate to the humerus bone; one or more hook-arm receivers in the bone plate; and one or more attachable hook-arms comprising one or more prongs and a device for attachment to the bone plate.

In some embodiments, the fractured greater tuberosity comprises a complete fracture having one or more separated fragments. In some embodiments, the fractured greater tuberosity comprises an incomplete fracture.

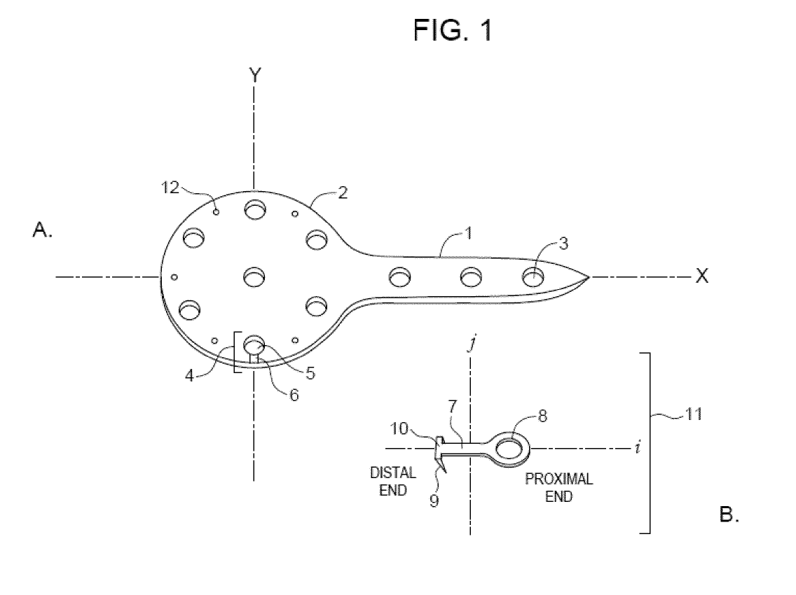

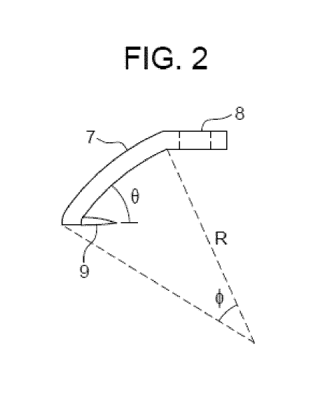

FIG. 1A shows an embodiment of a bone plate capable of fixating a fractured greater tuberosity. In one embodiment, the bone plate comprises a body portion 1 at a first end attached to a head portion 2 at a second end of the bone plate, one or more apertures 3 for fixing the bone plate to bone, and one or more receivers 4 on the bone plate for a hook-arm. The bone plate can also comprise one or more suture fixation sites 12. An exemplary hook-arm 11 is shown in FIG. 1B and FIG. 2. The hook-arm receiver 4 on the bone plate can overlap one of the apertures. In one embodiment the receiver includes a circular slot cavity 5 and a channel 6 to serve as a setting for the hook-arm attachment device. In some embodiments, the depth of the circular slot cavity 5 and channel 6 will match the depth of the hook-arm attachment device 8 and elongated shaft 7 to allow for the hook-arm and bone plate to be flush to one another when fixed in place.

Referring to FIG. 1, in some embodiments, the elongated body 1 has a curvature along its central axis (x-axis in FIG. 1), to contour to a typical humerus shaft. In some embodiments, all of the edges on the bone plate taper to contour to minimize the transition between the bone and the bone plate. In some embodiments, the enlarged head portion 2 contours to the greater tuberosity. In some embodiments, the head portion 2 has a shape that is approximately circular or rounded. In some embodiments, the head portion is ellipsoidal or polygonal in shape.

In some embodiments, the bone plate is oriented on the humerus bone so that the head portion is oriented more proximal to or on the greater tuberosity and the body portion of the bone plate extends more distal to the greater tuberosity, oriented on or towards the shaft of the humerus.

In some embodiments, the body portion has an elongated shape. In some embodiments, the width of the body is narrow relative to the width of the head portion. In some embodiments, the head width is enlarged, relative to the width of the body.

Depending on the patient and the fracture, the shape of the bone plate can vary. In some embodiments, the bone plate comprises an elongated body portion, without a head portion. In such embodiments, the head portion may not be necessary to repair the fracture. In some embodiments, the hook-arm receiver can be located on one end of an elongated body. In embodiments where the bone plate does not comprise an enlarged head, the elongated body can have two or more bone screw apertures to allow for proper fixation.

In some embodiments, the total length of the bone plate is from about 4 to about 12 centimeters (cm) long. In some embodiments, the length is about 6 cm to about 8 cm long. In some embodiments, the bone plate is about 4 cm, about 5 cm, about 6 cm or about 7 cm in length. In some embodiments, the elongated body portion is at least about 3 cm long, at least about 4 cm long, or at least about 5 cm long. In some embodiments, the elongated body of the bone plate has a width that is about 0.5 cm to about 1.3 cm. In some embodiments, the head portion is from about 2 cm to about 4 cm in length and has a width of from about 1 cm to about 2.5 cm. In some embodiments, the bone plate has a thickness or depth of from about 0.1 cm to about 0.6 cm.

In some embodiments, the bone plate has a length of about 7 cm, wherein the bone plate comprises a head portion that is rounded or circular in appearance that is about 3 cm long and the remainder is the elongated body portion; the bone plate has a thickness of about 0.4 cm, the width at the head portion at its widest point is about 2 cm and the width of the elongated body portion is about 1 cm.

In some embodiments, the bone plate and/or hook-arm is formed from a medical grade metal, such as a titanium alloy. In some embodiments, the bone plate and/or hook-arm is formed from the titanium alloy TI-6AL-4V. In some embodiments, bone screws are used to fix the bone plate to the bone. In some embodiments, the bone screws are made from a metal alloy, such as cobalt chrome (CoCr) alloy.

Apertures

The bone plate comprises one or more apertures extending through the upper and bone contacting surfaces of the bone plate. In some embodiments, the aperture includes a threaded portion. In some embodiments, the entire interior of the aperture is threaded. In some embodiments, the aperture includes a threaded portion and a non-threaded portion. In some embodiments, the threaded portion of the aperture is dimensioned and configured to engage a threaded head of a bone screw, and fix the bone screw at a predetermined angle with respect to the bone plate. In some embodiments, the threaded portion extends through the full thickness of the bone plate, i.e., from the upper surface to the bone contacting surface, thus maximizing the stability of the bone screw to bone plate interface.

In some embodiments, the body portion or the head portion or both the body and head portions of the bone plate comprise one or more apertures to fix the bone plate to the humerus bone. The number of apertures on the bone plate is not critical, as long as the bone plate is capable of being relatively fixed in place. In some embodiments, the number of apertures is 1, 2, 3, 4, 5, 6, 7, 8, 9, 10, 11, 12, 13, 14 or 15 or more. In some embodiments, the head has from 1 to 8 apertures. In some embodiments, it has from 4 to 7 apertures. In some embodiments, the head has 1, 2, 3, 4, 5, 6, 7, or 8 apertures. In some embodiments, the apertures can also function as receivers for the hook-arm, and therefore serve the dual purpose of fixing the bone plate to the bone and fixing the hook-arm to the bone plate. In some embodiments, a single aperture can accommodate both a bone screw and a hook-arm.

In some embodiments of the invention, the body of the bone plate comprises two or more bone screw apertures and the head portion has seven bone screw apertures. In some embodiments, one aperture is centrally located on the bone plate head while the remaining six apertures are evenly spaced around the outer edge of the head.

In one embodiment, referring to FIG. 1, the elongated body portion 1 has at least three approximately evenly spaced bone screw apertures 3 and the enlarged head has seven apertures. One bone screw aperture of the enlarged head is located approximately in the center of the head, one is located on the outer edge of each side of the middle aperture, and the remaining four are approximately evenly spaced above and below the middle three.

Hook-Arm Receiver

In accordance with the invention, the bone plate comprises one or more receivers for the hook-aim. As indicated above, an aperture can also function as a receiver. In some embodiments, the one or more receivers are located on the head portion. In some embodiments, the one or more receivers are located on the body portion. In some embodiments, both the head and body portions have one or more receivers for the hook-arm. Having multiple receivers on the bone plate can enable fixation of the greater tuberosity at or very near to the location of the fracture. In some embodiments, the fracture requires more than one hook arm to secure it. For example, in one embodiment, two adjacent receivers in close proximity are each attached with a hook-arm to secure a single fracture. In some embodiments, multiple fractures are present, and multiple hook aims are attached to multiple receivers on the bone plate to secure the fractures.

In some embodiments, the hook-arm receiver is as shown in FIG. 1. In this embodiment, the hook-arm receiver 4 on the bone plate can overlap one of the apertures. In one embodiment, the receiver includes a circular slot cavity 5 and a channel 6 to serve as a setting for the hook-arm attachment device. In some embodiments, the depth of the circular slot cavity 5 and channel 6 will match the depth of the hook arm attachment device 8 and elongated shaft 7 to allow for the hook-arm and bone plate to be flush to one another when in fixed in place.

Hook-Arm

In accordance with the invention, the bone plate comprises a hook-arm capable of securing a fracture to the greater tuberosity. In some embodiments, referring to FIG. 1 to aid in the identification of parts of the hook-arm, the hook-arm 11 comprises an elongated first shaft 7 having one or more prongs 9 at the distal end of the shaft and an attachment mechanism or device 8 to the bone plate on the proximal end of the shaft. In some embodiments, the one or more prongs 9 are attached directly to the elongated shaft while in other embodiments, the one or more prongs are indirectly attached to the elongated shaft 7. In some embodiments, the prongs project from a second shaft 10 located at the distal end of the elongated first shaft 7. In some embodiments, the second shaft 10 is oriented perpendicularly to the elongated first shaft 7. The length of the elongated shaft can vary. In some embodiments, the length of the elongated shaft 7 ranges from about 1 to about 2.5 cm, or from about 1 to about 1.5 cm in length. The length of the second or perpendicular shaft 10 to which the one or more prongs 9 are attached can vary depending on patient size and/or fracture depth, and in some embodiments the length is about 0.3 cm to about 1 cm. In some embodiments, the width and the depth or thickness of the perpendicular shaft 10 will match that of the elongated shaft to which it is attached. The depth or thickness of the elongated and perpendicular shaft can vary, and in some embodiments, the depth or thickness is between about 0.1 and about 0.4 cm.

The length of the prongs 9 can vary. In some embodiments, the one or more prongs 9 will be between about 2 to about 4 mm in length and about 1 to about 2 mm in width. The spacing between the prongs can vary, and in some embodiments, the spacing between the prongs is from about 0.2 cm to about 1 cm. In some embodiments, the hook-arm has two prongs. In some embodiments, the hook-arm has 3, 4, 5, 6 or 7 prongs. In some embodiments, the ends of the one or more prongs that contact bone can be tapered to help grasp the bone fragment. In some embodiments, barbs can be located on the one or more prongs to provide for additional grip. In some embodiments, the hook-arm shaft can have a curvature along the central axis, j, as depicted in FIGS. 1 and 2, which, in some embodiments can approximately match the curvature of the fracture site. In some embodiments, the hook-arm shaft can have a curvature, along the central axis, j, to allow for abutment against the greater tuberosity and humeral head. In some embodiments, the one or more prongs are oriented at an angle θ, as shown in FIG. 2, of less than or equal to about 90° to the distal end of the hook-arm shaft. In some embodiments, the angle is about 20°, about 30°, about 40°, about 50°, about 60°, about 70°, about 80° or about 90°. In some embodiments, the angle θ is not less than about 15°. In some embodiments, the angle θ is between about 15° and about 60°.

Various attachment methods can be employed to attach the hook arm attachment device to the receiver on the bone plate. For example, attachment methods can include, but are not limited to set screws that do not enter the bone, snap in pieces, latches, grooves, or hooks. Other mechanisms to attach the hook-arm to the bone plate can also be used. Other mechanisms for attachment do not deviate from the spirit of the invention and should be considered as being embodied herein.

In some embodiments, the hook-arm can be attached to the bone plate on a track or other mechanism that will allow for it to extend out to grasp onto the greater tuberosity fragment and then fix it in place. The hook-arm can also be attached by a hinge or latching mechanism. The specific mechanisms for the attached hook-arm to grasp and fixate a fragment are intended to be illustrative by example and should not be limiting.

In some embodiments, the attachment device is a ring system, located at the proximal end of the hook-arm. The hook-arm attachment device 8 of FIG. 1 employs a ring having a circular shape that can be inserted into a circular cavity 5 on the bone plate. In some embodiments, the circular cavity is located around one of the apertures of the bone plate and the hook-arm is flush with the rest of the bone plate when it is inserted. In some embodiments, the ring can have an inner circumference of about 1.5 mm to about 3 mm, in some embodiments, about 2.5 mm, and an outer circumference of about 2 mm to about 5 mm. In some embodiments, the ring can fit closely into a receiver on the bone plate. In some embodiments, the ring is placed into an aperture on the bone plate. In some embodiments, the ring is secured in place with a bone screw that is inserted through the ring and screw aperture. In some embodiments, the hook-arm ring is like a washer between the bone screw head and the bone screw aperture.

In some embodiments, the attachment device is similar to the ring system described above, except that the shape of the attachment device is not circular like a ring. For example, it can be of any geometrical shape, such as polygonal, provided that it fits closely to the receiver or aperture on the bone plate and can be secured in place.

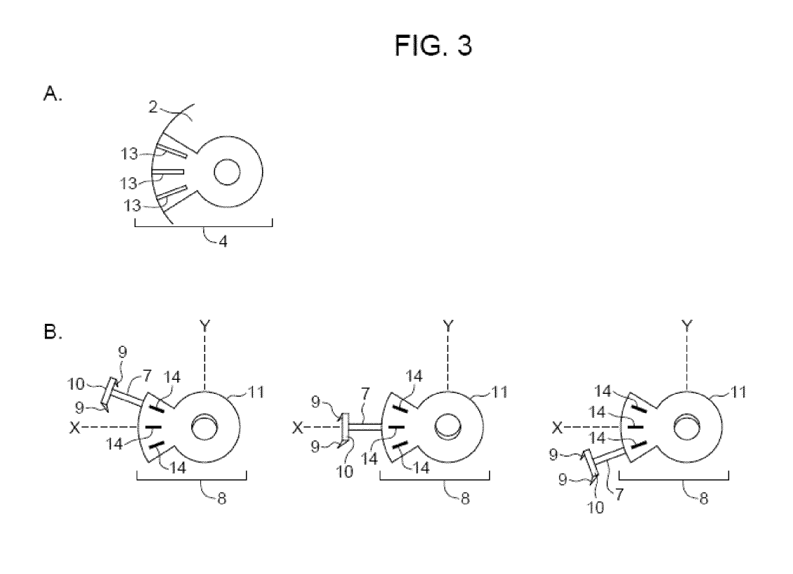

In some embodiments, the hook-arm can be attached to the bone plate using a tongue and groove joint system. An embodiment of the invention that utilizes a tongue and groove system is shown in FIG. 3. In tongue and groove attachment schemes, for example, the bone plate can comprise one or more grooves 13 that fit one or more ridges or tongues 14. In some embodiments, the tongue projects a little less than the groove is deep, and the two pieces thus fit together closely. The tongues or grooves can be on either the hook-arm or the bone plate. In some embodiments, the hook-arm is fixed to the bone plate by 1, 2, 3, 4, 5, 6, 7, 8, 9, or 10 tongues and grooves. In some embodiments, a tongue and groove system is used in combination with another attachment system, such as the ring system discussed above.

In some embodiments, the bone plate is specifically contoured for use on only one of the left or right humerus bones. In some embodiments, the bone plate can be used on either the left or right humerus, and is not specifically contoured to fit only one of the bones. For example, the bone plate can be configured to secure a similar fracture on either the right or left humerus by placing the receiver on opposite sides of the bone plate. Similar adjustments can be made to the head and/or body portion so that it contours properly to either the left or right humerus. In some embodiments, the head contains more than one hook-arm receivers. In one embodiment, the head comprises a hook-arm receiver on either side of the head to enable the bone plate to be used on greater tuberosity fractures on either the left or right humerus.

The invention also provides methods for stabilizing a fractured greater tuberosity in a subject using the bone plates of the invention. In some embodiments, the method comprises attaching a bone plate of the invention to the humerus of a subject having a fractured greater tuberosity; making one or more holes into the greater tuberosity; securing the one or more prongs from the hook-arm into the holes; inserting the hook-arm attachment device into the hook-arm receiver; and securing the hook-arm to the bone plate, thereby stabilizing the fractured greater tuberosity.

The body of the bone plate can be attached to the humerus by any method currently known in the art. In one embodiment, the body of the bone plate is first attached to the humerus using two or more bone screws. One or more pilot holes can be made in the greater tuberosity fragment. The number of pilot holes and their location will depend on the number of prongs and their orientation on the hook-arm. The diameter of the pilot holes should be less than or equal to the diameter of the one or more hook-arm prongs. The hook-arm prongs are then inserted into the pilot holes and the hook-arm attachment device, such as a ring, is placed into the receiver on the head portion. A bone screw can then be inserted through the hook-arm ring, through the bone screw aperture, and fixed to the humeral head. The type of screw can vary depending on the structure and condition of the bone.

The present invention also provides kits comprising the bone plates of the invention, and optionally instructions for their use. In some embodiments, the kit comprises combinations of the components described above. In some embodiments, the kit comprises one bone plate and a set of hook-arms, such as 2, 3, 4, 5, 6, 7, 8, 9, or 10 hook-arms. The hook-arms can be of different shapes and sizes, for example, they can have different elongated body lengths, different head sizes, different configurations of the apertures, and different positions for the receiver. In some embodiments, the bone plates share the same attachment mechanism. In some embodiments, the kit comprises more than one bone plate of various lengths and having different numbers of apertures, for example, from 3-12 apertures. In some embodiments, the kit comprises bone plates that are configured for both the left and right humerus.

The preceding drawings and description merely explain the present invention by way of example and are not intended to be limiting. While certain embodiments and features have been disclosed herein, a person having ordinary skill in the art having this disclosure would be able make changes and alterations. It is intended that the appended claims cover all such changes and alterations as fall within the scope of such claims and that the claims not be limited to or by such preferred embodiments or features.

All references, including publications, patent applications, and patents, cited herein are hereby incorporated by reference to the same extent as if each reference were individually and specifically indicated to be incorporated by reference and were set forth in its entirety herein.

What is claimed is:

1. A bone plate for stabilizing a fractured greater tuberosity, comprising:
i) one or more apertures for fixing the bone plate to bone;
ii) one or more hook-arm receivers; and
iii) one or more attachable hook-arms comprising one or more prongs and a device for attachment to the bone plate.

2. The bone plate of claim 1, further comprising a body portion at a first end and a head portion at a second end, wherein the body has an elongated shape.

3. The bone plate of claim 2, wherein the head portion has a width that is enlarged relative to the body portion.

4. The bone plate of claim 2, wherein the head portion is rounded in shape.

5. The bone plate of claim 2, wherein the head and body portions both comprise one or more apertures for fixing the bone plate to bone and the one or more hook-arm receivers is in the head portion.

6. The bone plate of claim 1, wherein the one or more apertures are configured to accept a bone screw.

7. The bone plate of claim 2, wherein the one or more prongs and device for attachment to the head is separated by an elongated shaft.

8. The bone plate of claim 1, wherein the attachment device is in the shape of a ring and the hook-arm receiver is a circular cavity around one of the apertures.

9. The bone plate of claim 2, wherein the head of the bone plate contains one or more grooves for the hook-arm to be fixated.

10. The bone plate of claim 1, wherein the hook-arm has two prongs.

11. The bone plate of claim 1, further comprising one or more suture fixation locations on the bone plate.

12. A bone plate for stabilizing a fractured greater tuberosity comprising:
an elongated body at a first end;
an enlarged head at a second end;
at least one bone screw aperture in the first and second end; and
an attached hook-arm extending from the enlarged head having one or more prongs.

13. A method for stabilizing a fractured greater tuberosity in a subject comprising:
i) attaching the bone plate of claim 1 to the humerus of a subject having a fractured greater tuberosity;
ii) making one or more holes into the greater tuberosity;
iii) securing the one or more prongs from the hook-arm into the holes of ii);
iv) inserting the hook-arm attachment device into the hook-arm receiver; and
v) securing the hook-arm to the bone plate, thereby stabilizing the fractured greater tuberosity.

14. The method of claim 13, wherein the fractured greater tuberosity comprises a complete fracture having one or more separated fragments.

15. The method of claim 13, wherein the fractured greater tuberosity comprises an incomplete fracture.

16. A kit comprising the bone plate of claim 1.

17. The kit of claim 16, comprising a plurality of hook-arms.

18. The kit of claim 16, comprising a plurality of bone plates.

19. The kit of claim 16, comprising a plurality of bone plates and a plurality of hook arms of different sizes.
